(19) United States
(12) Patent Application Publication
(10) Pub. No.: US 2004/0081994 A1
(43) Pub. Date: Apr. 29, 2004

(54) Biochemical methods for measuring metabolic fitness of tissues or whole organisms

(76) Inventor: Marc K. Hellerstein, Kensington, CA (US)

Correspondence Address: MORRISON & FOERSTER LLP, 425 MARKET STREET, SAN FRANCISCO, CA, 94105-2482, US

(21) Appl. No.: 10/664,513

(22) Filed: Sep. 16, 2003

Related U.S. Application Data
(60) Provisional application No. 60/411,029, filed on Sep. 16, 2002.

(51) Int. Cl. C12Q 1/68; A61K 49/00; A61K 51/00

(52) U.S. Cl. 435/6; 424/1.11; 424/9.1

(57) ABSTRACT

The present invention relates to biochemical methods for assessing metabolic fitness and/or aerobic demands of a living system. Specifically, the rate of synthesis and turnover of the molecular components of mitochondrial mass are used to determine the aerobic capacity and/or aerobic demand of tissues or living organisms. The direct measurement of metabolic fitness and/or aerobic demand by this means can be used as an index of the efficacy of an exercise training program or other therapeutic intervention; as a medical risk factor for predicting the risk of cardiovascular disease, diabetes, death or other health outcome; or as an aid to pharmaceutical companies for drug discovery in the area of metabolic fitness, deconditioning, and oxidative biology.

### CROSS-REFERENCE TO RELATED APPLICATIONS

This application claims priority to 60/411,029 filed on Sep. 16, 2002, which is hereby incorporated by reference in its entirety.

### FIELD OF THE INVENTION

The present invention is directed to the field of oxidative biology. In particular, methods for determining metabolic fitness by measuring the synthesis rates of mitochondrial DNA, RNA, proteins, or phospholipids are described.

### BACKGROUND OF THE INVENTION

The level of physical fitness (metabolic fitness, cardiorespiratory fitness) in humans has been shown to be a strong predictor for heart disease, diabetes, and overall mortality. Recent epidemiologic studies suggest that physical fitness instead of body fatness may be the most accurate risk factor in predicting all-cause mortality (Blair et al., Changes in Physical Fitness and All-Cause Mortality, JAMA 273(14):1093-1098 (1998) and Lee et al., Cardiorespiratory Fitness, Body Composition, and All-Cause and Cardiovascular Disease Mortality in Men, Am J Clin Nutr 69(3):373-380 (1999)). Support for this conclusion is evidenced by data demonstrating that some individuals who are overweight but fit metabolically exhibit a better health prognosis than individuals who are thin but unfit metabolically (see Lee et al., supra). Thus, being overweight may primarily serve as a marker for an underlying sedentary lifestyle and metabolically unfit state, rather than being the true risk-factor itself.

These findings have potentially profound clinical and public health implications. A physician's focus on the body fat of a patient may be misplaced if the key variable to monitor is metabolic fitness. Similarly, pharmaceutical companies looking for drugs that improve health might be better advised to work on agents that increase tissue oxidative (aerobic) capacity than on agents that reduce body fat content. However, currently available methods for assessing the metabolic fitness of whole organisms, e.g., exercise testing, are crude, non-biochemical, poorly reproducible, and difficult to perform.

For example, exercise testing requires an individual to exercise on equipment such as a treadmill or stationary bike, with continuous electrocardiographic and blood pressure monitoring. Typically, exercise is continued under a controlled program until the individual is unable to continue or until 85% of the individual's maximal heart rate is achieved (Hutter, A. M., Jr. (1991). “Ischemic Heart Disease: Angina Pectoris,” Section 1 In Scientific American Medicine. E. Rubenstein and D. D. Federman eds., Scientific American, Inc., p. 4). With such a protocol, it can be easily seen that numerous factors including mental illness, physical impairments due to such afflictions as respiratory or muscle disease, and inconsistent physical effort by the patient may affect test results. Moreover, there is some potential risk associated with this protocol (i.e., the exertion required). Furthermore, exercise testing is characterized by wide inter-observer variability (due to differences in supervisors' performance and difficulty in standardization) and use of bulky equipment that is not easily stored in a medical office.

Therefore, new methods that are more convenient for outpatient use and which objectively and reliably determine metabolic fitness are needed.

### SUMMARY OF THE INVENTION

In order to meet these needs, the present invention provides methods of assessing metabolic fitness or aerobic demand of a living system. In one aspect, a method is disclosed for assessing metabolic fitness or aerobic demand of a living system by administering an isotopically labeled precursor molecule to the living system time sufficient for the label of the isotopically labeled precursor molecule to be incorporated into a mitochondrial molecule; obtaining one or more mitochondrial molecules from the living system; measuring the isotopic content, isotopic pattern, rate of change of isotopic content, or rate of change of isotopic pattern of the mitochondrial molecule; and calculating the rate of synthesis or degradation of the mitochondrial molecule to assess metabolic fitness or aerobic demand of the living system. In one variation, the isotopically labeled precursor molecule is labeled with a stable isotope. In another variation, the isotopically labeled precursor may be one or more of ²H-labeled glucose, ¹³C-labeled glucose, a ²H-labeled amino acid, a ¹⁵N-labeled amino acid, a ¹³C-labeled amino acid, ²H-labeled acetate, ¹³C-labeled acetate, a ²H-labeled ribonucleoside, a ¹³C-labeled ribonucleoside, a ¹⁵N-labeled ribonucleoside, a ²H-labeled deoxyribonucleoside, a ¹³C-labeled deoxyribonucleoside, a ¹⁵N-labeled deoxyribonucleoside, a ²H-labeled fatty acid, and a ¹³C-labeled fatty acid. In a further variation, the isotopically labeled precursor molecule is ²H₂O. The isotopically labeled precursor molecule may also be ¹³C-glycine.

In another variation, the label is a radioactive isotope. In another variation, the isotopically labeled precursor molecule may be one or more of ³H-labeled glucose, ¹⁴C-labeled glucose, a ³H-labeled amino acids, a ¹⁴C-labeled amino acid, ³H-labeled acetate, ¹⁴C-labeled acetate, a ³H-labeled ribonucleoside, a ¹⁴C-labeled ribonucleoside, a ³H-labeled deoxyribonucleoside, a ¹⁴C-labeled deoxyribonucleoside, a ³H-labeled fatty acid, and a ¹⁴C-labeled fatty acid.

In a further variation, the mitochondrial molecule may be any molecular or macromolecular component of a mitochondrion. Examples of mitochondrial molecules include a DNA molecule, an RNA molecule, a protein, or a lipid. In one variation, the mitochondrial molecules is an RNA molecule, which in a further variation may be one or more ribosomal RNA, transfer RNA, or messenger RNA. In another variation, the mitochondrial molecule may be a protein such as a subunit of cytochrome c oxidase, a subunit of F₀ ATPase, a subunit of F₁ ATPase, a subunit of cytochrome c reductase, or a subunit of NADH-CoQ reductase. In an additional variation, the mitochondrial molecule may be a lipid, such as a phospholipid. In an additional aspect, the phospholipids may be one or more of a cardiolipin, phosphatidylcholine, phosphatidylethanolamine, or mixture thereof.

In another aspect, the living system is a tissue. Variations of tissues include muscle tissue such as skeletal muscle and cardiac muscle, and adipose tissue.

The living system may also be an animal. The animal may be a mammal. The mammal may be a rodent. The mammal may be a human.

The living system is a cell. In a further aspect, the cell is a platelet. In another variation, the cell may be a cultured cell in a high-throughput screening assay system.

In a further aspect, the step of measuring isotopic content, pattern or rate of change of isotopic content, or pattern may be performed by mass spectroscopy, NMR spectroscopy, or liquid scintillation counting.

In another variation, the isotopically labeled precursor molecule is administered orally.

In another aspect, the methods are directed to identifying a drug agent capable of altering metabolic fitness or aerobic demand of a living system. In one variation, the method includes assessing the metabolic fitness or aerobic demand of the living system, administering the drug agent to the living system; and assessing the metabolic fitness or aerobic demand of the living system, wherein a change in the metabolic fitness or aerobic demand of the living system before and after administration of the drug agent identifies the drug agent as capable of altering the metabolic fitness or aerobic demand of the living system. In another variation, the method includes assessing the metabolic fitness or aerobic demand of a first the living system, wherein the drug agent has not been administered to the first living system; assessing the metabolic fitness or aerobic demand of a second the living system to which the drug agent has not been administered, and comparing the metabolic fitness or aerobic demand in the first and second living systems, wherein a change in the metabolic fitness or aerobic demand of the first and second living systems identifies the drug agent as capable of altering the metabolic fitness or aerobic demand of the living system. The living system may be a mammal, such as a human or a rodent. The living system may be a cell, such as a cultured cell in a high-throughput screening assay system. In a further variation, the isotopically labeled precursor molecule is contacted with cell culture media. In an additional variation, the drug agent is tested for the ability to prevent deconditioning of a living system. In a still further variation, drug agent is tested for the ability to increase metabolic fitness or aerobic demand in response to an exercise or other training regimen. In an additional variation, the present invention is also directed to previously identified drug agents.

In a further aspect, the present invention is directed to kit for assessing the metabolic fitness of a living system. The kit may include one or more isotopically labeled precursor molecules and instructions for use of the kit. In another variation, the kit may include further including a tool for administering the isotopically labeled precursor molecule. In a further variation, the kit may also include an instrument for obtaining a sample from the subject. In a still further variation, the isotopically labeled precursor molecule is isotopically labeled water.

In another aspect, the present invention is directed to an isolated isotopically perturbed mitochondrial DNA, isolated isotopically perturbed isolated cardiolipin, one or more isolated isotopically perturbed mitochondrion, or one or more isotope-labeled precursor molecule. In another aspect, the present invention is directed to an isolated isotope-labeled mitochondrial molecule made by administering an isotope-labeled precursor molecule to the host organism for a period of time sufficient for an isotope label of the isotope-labeled precursor molecule to become incorporated into a mitochondrial molecule.

### BRIEF DESCRIPTION OF THE DRAWINGS

FIG. 1A is an exemplary schematic of the protocol for isotopically labeled water (²H₂O) administration and sample collection for rats.

FIG. 1B illustrates the protocol for isotopically labeled water (²H₂O) administration and sample collection for human subjects.

FIG. 2A shows the increased incorporation of ²H from administered ²H₂O into mitochondrial DNA isolated from rats subjected to one week of exercise training as measured by gas chromatography/mass spectrometry.

FIG. 2B demonstrates the incorporation of ²H into mitochondrial DNA isolated from human muscle biopsies as measured by gas chromatography/mass spectrometry.

FIG. 3A shows the experimental protocol for the measurement of the rate of synthesis of mitochondrial DNA and mitochondrial phospholipids in human subjects, as measured from mitochondria isolated from muscle biopsies taken after the human subjects ingested ²H₂O.

FIG. 3B shows the effects of different exercise regimens on incorporation of ²H from administered ²H₂O into mitochondrial phospholipids.

FIG. 4A shows the increased incorporation of ²H from administered ²H₂O into cardiolipin (CL), phosphatidylcholine (PL), and phosphatidylethanolamine (PE) in mitochondria isolated from the hindlimb muscle of rats subjected to voluntary exercise.

FIG. 4B shows the increased incorporation of ²H from administered ²H₂O into cardiolipin (CL), phosphatidylcholine (PL), and phosphatidylethanolamine (PE) in mitochondria isolated from the heart muscle of rats subjected to chronic exercise.

FIG. 5 depicts the average cytochrome C oxidase subunit IV expression in hindlimb muscle from rats trained for 1, 2, and 6 weeks (n=6 per time point) compared to controls (n=6 per time point). Data are ±S.D. * denotes statistical significance (p<0.05) versus control values.

FIG. 6 depicts Cytochrome C oxidase subunit IV expression from rats detrained for 4 weeks (n=6) compared to controls (n=6). Data are ±S.D. No significant differences between groups are present.

### DETAILED DESCRIPTION OF THE INVENTION

The present invention provides methods for the biochemical assessment of metabolic fitness by measuring the rate of mitochondrial synthesis or degradation of mitochondrial molecules such as deoxyribonucleic acids (DNA), ribonucleic acids (RNA), proteins, or lipids in mitochondria of tissues. The rate of synthesis or degradation is based on the isotopic content and/or pattern or the rate of change of the isotopic content and/or pattern in mitochondrial molecules measured after administration of, or contact with, one or more isotopically labeled precursor molecules, including isotopically labeled water, where the isotope label is incorporated into mitochondrial molecules.

### General Techniques

Practice of the present invention will generally utilize, unless otherwise indicated, conventional techniques of molecular biology, microbiology, cell biology, biochemistry, and immunology, which are within the skill of the art. Such techniques are fully explained in the literature, for example, in Cell Biology: A Laboratory Notebook (J. E. Cellis, ed., 1998); Current Protocols in Molecular Biology (F. M. Ausubel et al., eds, 1987); Short Protocols in Molecular Biology (Wiley and Sons, 1999); Mass Isotopomer Distribution Analysis: A Technique for Measuring Biosynthesis and Turnover of Polymers (Hellerstein et al., Am J Physiol 263 (Endocrinol Metab 26):E988-E1001 (1992)); and Mass Isotopomer Distribution Analysis at Eight Years: Theoretical, Analytic, and Experimental Considerations (Hellerstein et al., Am J Physiol 276 (Endocrinol Metab 39):E1146-1170 (1999)). Furthermore, procedures employing commercially available assay kits and reagents will typically be used according to manufacturer defined protocols unless otherwise noted.

### Definitions

The terms “metabolic fitness”, “physical fitness”, and “cardiorespiratory fitness” herein are used interchangeably, and refer to the capacity for oxidative metabolism or aerobic activity of a living system.

By “living system” is meant herein any living entity including a cell, cell line, tissue, organ, and organism. The living system is preferably an organism. Examples of organisms include any animal, preferably a vertebrate, more preferably a mammal, most preferably a human. Examples of mammals include nonhuman primates, farm animals, pet animals, for example cats and dogs, and research animals, for example mice, rats, and humans. The human can be healthy or suffering from, or diagnosed with, a disease or disorder.

“Aerobic demand” refers to the oxidative needs imposed on a cell, tissue, or organism in vivo.

By “isotopes” it is meant herein atoms with the same number of protons and hence the same element but with different numbers of neutrons (e.g., ¹H vs. ²H or ³H). As is commonly known in the art, the symbol “D” is used interchangeably with the symbol ²H to refer to deuterium.

“Isotopically labeled precursor molecule” and “isotope labeled precursor molecule” are used interchangeably and refer to any isotope labeled precursor molecule from which the isotope label into a mitochondrial molecule in a living system. Examples of isotope labeled precursor molecules include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition, therefore each isotopologue has a unique exact mass but may riot have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as M₀; for most organic molecules, this is the species containing all ¹²C, ¹H, ¹⁶O, ¹⁴N, etc. Other mass isotopomers are distinguished by their mass differences from M₀ (M1, M2, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonymously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m0 and the masses of other mass isotopomers are identified by their mass differences from m0 (m1, m2, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted).

“Isolating” refers to separating one component from one or more additional components in a mixture of components. For example, isolating a biochemical component refers to separating one biochemical components from a mixture of biochemical components. Small quantities of additional biochemical components may be present in the isolated biochemical component.

As used herein, the terms “precursor subunit,” “precursor molecule,” and “precursor” are used interchangeably to refer to the metabolic precursors used during polymeric synthesis of specific molecules. Examples of precursor subunits include acetyl CoA, ribonucleic acids, deoxyribonucleic acids, amino acids, glucose, and glycine.

“Labeled water” as used herein refers to water that contains isotopes. Examples of labeled water include ²H₂O, ³H₂O, and H₂¹⁸O. As used herein, the term “isotopically labeled water” is used interchangeably with “labeled water.”

“Isotopic content” refers to the content of isotopes in a molecule or population of molecules relative to the content in the molecule or population of molecules naturally (i.e., prior to administration or contacting of isotope labeled precursor subunits). The term “isotope enrichment” is used interchangeably with isotopic content herein.

“Isotopic pattern” refers to the internal relationships of isotopic labels within a molecule or population of molecules, e.g., the relative proportions of molecular species with different isotopic content, the relative proportions of molecules with isotopic labels in different chemical loci within the molecular structure, or other aspects of the internal pattern rather than absolute content of isotopes in the molecule.

“Molecular flux rate” refers to the rate of synthesis and/or breakdown of molecules within a cell, tissue, or organism. “Molecular flux rate” also refers to a molecule's input into or removal from a pool of molecules, and is therefore synonymous with the flow into and out of said pool of molecules.

“Oxidative metabolism” refers to the sum total of all energy-yielding biochemical transformations of fuels by a cell, tissue, organism, or other living system that ultimately require the involvement of molecular oxygen interacting with the oxidative phosphorylation apparatus (electron transport chain or respiratory enzyme system) in the cell, tissue, or organism.

“Drug agent,” “pharmaceutical agent,” and “pharmacological agent” are used interchangeably to refer to any chemical entities, known drug or therapy, approved drug or therapy, biological agent (e.g., gene sequences, poly or monoclonal antibodies, cytokines, and hormones). Drug agents include, but are not limited to, any chemical compound or composition disclosed in, for example, the 13th Edition of The Merck Index (a U.S. publication, Whitehouse Station, N.J., U.S.A.), incorporated herein by reference in its entirety.

“Mitochondrial molecule” refers to a molecule, such as a macromolecule, of a mitochondrion. Examples of mitochondrial molecules include, but are not limited to, DNA, RNA, proteins, lipids, and carbohydrates. The mitochondrial molecule may be synthesized or degraded within a mitochondrion, synthesized or degraded outside the mitochondrion, or imported into, or exported from, a mitochondrion. If a mitochondrial molecule is imported into a mitochondrion, then the mitochondrial molecule may or may not be further processed once within a mitochondrial space. In like manner, once a mitochondrial molecule is exported from a mitochondrion, that mitochondrial molecule may or may not be further processed.

### Mitochondrial Adaptation to Aerobic Demand

Mitochondria are the organelles of oxidative phosphorylation and are present in nearly all eukaryotic cells. The mitochondrial mass (i.e., the sum of mitochondrial components, including DNA, RNA, proteins, lipids, and other mitochondrial molecules) within a cell depends upon the cell type and a variety of physiologic factors. Although large differences in mitochondrial mass have been documented for different cell types, under resting conditions, the mitochondrial mass of each particular cell type is characteristic of metabolic fitness. The mitochondrial mass generally reflects the capacity of a cell or tissue for oxidative metabolism or aerobic activity.

However, a change in aerobic demand, e.g., due to aerobic training placed upon a tissue such as skeletal or cardiac muscle, has been identified as varying mitochondrial mass. In general, mitochondrial mass increases in response to aerobic exercise training programs, for example, and decreases in response to the deconditioning that occurs with inactivity such as bedrest. This adaptability of mitochondrial mass to the aerobic demand placed upon a tissue, thereby modulating the capacity of a tissue for oxidative metabolism (its aerobic capacity), is a fundamental characteristic of oxidative biology. Mitochondrial adaptability has profound implications for human health in the setting of the progressively more sedentary lifestyles associated with industrialization and urbanization, as is occurring internationally.

There are several unique features about the biochemistry of adaptive changes in tissue mitochondrial mass (Attardi et al., Biogenesis of Mitochondria, Ann Rev Cell Biol 4:289-333 (1988)). First, mitochondrial DNA is separate and distinct from the remainder of eukaryotic cellular DNA, which is present in the nucleus. Additionally, the mitochondrial genome is circular rather than arranged linearly within chromosomes in the nucleus, is small (16-20 kB in animals) compared to nuclear DNA, is almost completely lacking in introns, is synthesized using a different DNA polymerase (DNA polymerase γ) than is present in the nucleus and is inherited maternally and independently of nuclear mitosis or meiosis. Moreover, mitochondrial DNA synthesis is linked to mitochondrial RNA synthesis: The former (DNA replication) depends upon priming by DNA-based RNA-transcription (Clayton D., Replication and Transcription of Vertebrate Mitochondrial DNA, Ann Rev Cell Biol 7:453-478 (1991)). This dependence of replication on transcription results in coordinate induction of increased mitochondrial DNA synthesis when the cell is signaling the need for more mitochondrial RNA synthesis. Finally, mitochondrial proteins and lipids are almost entirely derived from extra-mitochondrial synthesis, unlike mitochondrial DNA. Over 90% of mitochondrial proteins are synthesized from cytosolic messenger RNA templates which are in turn derived from nuclear DNA coding sequences. Proteins synthesized in the cytosol are then imported into mitochondria (see Lee et al. and Attardi et al., supra). Only a small number of (essential) enzymes of mitochondrial oxidative metabolism are coded by mitochondrial DNA. Most of the mitochondrial RNA transcripts derived from mitochondrial DNA are used for the protein synthetic apparatus (e.g., for ribosomal or transfer RNA), rather than for messenger RNA.

It should also be noted that the model that discrete mitochondria exist and that there is a countable “mitochondrial number” is increasingly believed to be an oversimplification and incorrect (Robin et al., Mitochondrial DNA Molecules and Virtual Number of Mitochondria Per Cell in Mammalian Cells, J Cell Physiol 136:507-513 (1988)). Mitochondria in a cell are connected three-dimensionally through a reticulum that probably allows the flow of materials among the components. Because mitochondrial DNA exists as small circular genomes that are present at many copies per apparent mitochondrial “unit,” even the DNA content of the mitochondrial reticulum is probably exchangeable between and among components.

The currently available techniques for measuring mitochondrial mass or activity are all limited in one findamental respect; i.e., they are static in nature rather than reflecting dynamic processes. Typically, these techniques measure levels of such factors as mitochondrial oxidative enzymes (e.g., citrate synthase) or mitochondrial DNA or RNA, which only reveals the concentration present at that moment in time. However, adaptations in mitochondrial mass in response to aerobic demands involve kinetic changes (i.e., changes in molecular flux rates, including the rates of synthesis or catabolism of mitochondrial components). There had been until recently, however, no way to assess the rates of synthesis or breakdown of mitochondrial components, and therefore, no way to assess the underlying dynamics of mitochondrial mass or the trajectory (the direction of change) of mitochondrial mass or mitochondrial dynamics in response to tissue oxidative demand. In one aspect, mitochondrial mass changes in response to the synthesis and/or degradation of mitochondrial molecules.

### Methods For Assessing Metabolic Fitness

The present invention provides methods for assessing metabolic fitness by measuring the rate of synthesis or degradation of various mitochondrial molecules. Examples of mitochondrial molecules include, but are not limited to DNA, RNA, lipids, carbohydrates, and proteins. RNA includes ribosomal RNA, transfer RNA, and messenger RNA. Lipids include phospholipids. Proteins include subunits of the various macromolecular complexes comprising the electron transport chain and involved in oxidative phosphorylation (aerobic respiration). These subunits include subunits of cytochrome c oxidase, subunits of F₀ ATPase, subunits of F₁ ATPase, subunits of cytochrome c reductase, and subunits of NADH-CoQ reductase.

In one aspect, a method is disclosed for assessing metabolic fitness or aerobic demand of a living system by administering an isotopically labeled precursor molecule to the living system time sufficient for the label of the isotopically labeled precursor molecule to be incorporated into a mitochondrial molecule; obtaining one or more mitochondrial molecules from the living system; measuring the isotopic content, isotopic pattern, rate of change of isotopic content, or rate of change of isotopic pattern of the mitochondrial molecule; and calculating the rate of synthesis or degradation of the mitochondrial molecule to assess metabolic fitness or aerobic demand of the living system.

### A. Administering to a Living System an Isotope-Labeled Precursor Molecule

1. Labeled precursor molecules

a. Isotope labels

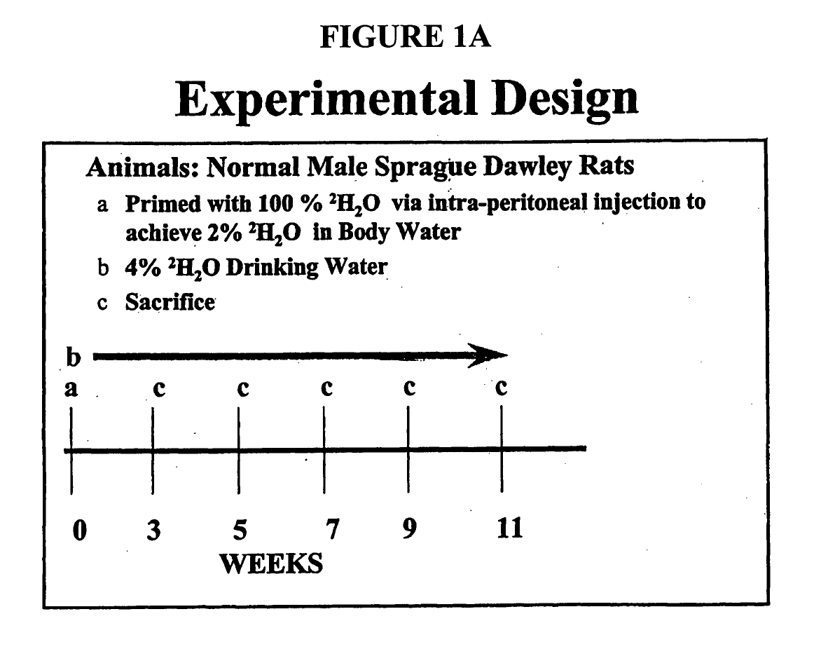

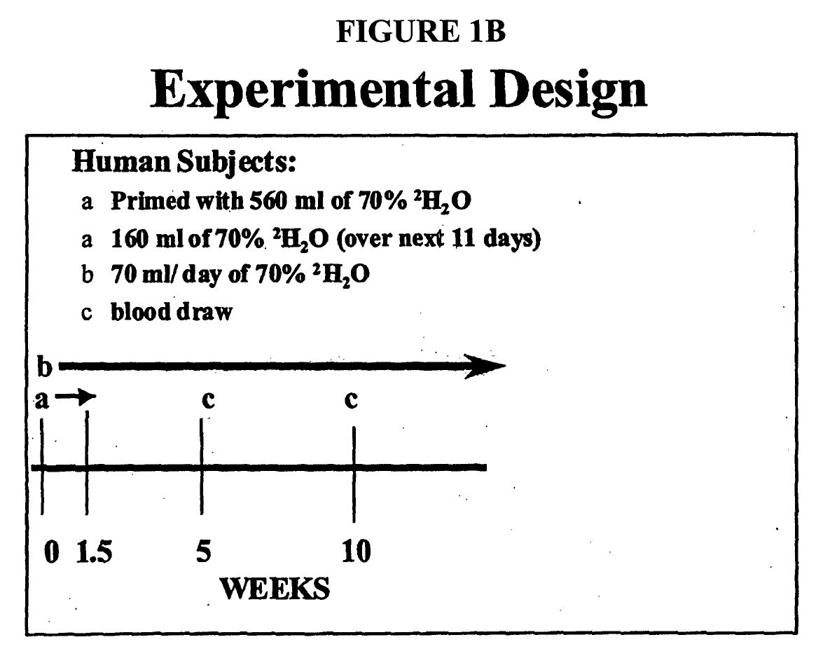

As illustrated in FIG. 1, the first step in measuring biosynthesis, breakdown, and/or turnover rates involve administering an isotope-labeled precursor molecule to a living system. The isotope labeled precursor molecule may be a stable isotope or radioisotope. Isotope labels that can be used include, but are not limited to, ²H, ¹³C, ¹⁵N, ¹⁸O, ³H, ¹⁴C, ³⁵S, ³²P, ¹²⁵I, ¹³¹I, or other isotopes of elements present in organic systems.

In one embodiment, the isotope label is ²H.

b. Precursor Molecules

The precursor molecule may be any molecule that is metabolized in the body to form a mitochondrial molecule. Isotope labels may be used to modify all precursor molecules disclosed herein to form isotope-labeled precursor molecules.

The entire precursor molecule may be incorporated into one or more mitochondrial molecules (e.g., mitochondrial molecules). Alternatively, a portion of the precursor molecule may be incorporated into one or more mitochondrial molecules.

Precursor molecules may include, but are not limited to, CO₂, NH₃, glucose, lactate, H₂O, acetate, fatty acids.

i. Water as a Precursor Molecule

Water is a precursor of proteins, polynucleotides, lipids, carbohydrates, modifications or combinations thereof, and other mitochondrial molecules. As such, labeled water may serve as a precursor in the methods taught herein.

Labeled water may be readily obtained commercially. For example, ²H₂O may be purchased from Cambridge Isotope Labs (Andover, Mass.), and ³H₂O may be purchased, e.g., from New England Nuclear, Inc. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 liters water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved using the techniques of the invention. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. Applicants have discovered that as long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

Labeled water may be used as a near-universal precursor for most classes of mitochondrial molecules.

ii. Protein, Oligo/Polynucleotide, Lipid, and Carbohydrate Precursors

In another embodiment, precursor molecules are precursors of proteins, polynucleotides, lipids, and carbohydrates.

(a) Precursors of Proteins

The precursor molecule may be any protein precursor molecule known in the art. These precursor molecules may be CO₂, NH₃, glucose, lactate, H₂O , acetate, and fatty acids.

Precursor molecules of proteins may also include one or more amino acids. The precursor may be any amino acid. The precursor molecule may be a singly or multiply deuterated amino acid. The precursor molecule is one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted with non-deuterated amino acids. All isotope labeled precursors may be purchased commercially, for example, from Cambridge Isotope Labs (Andover, Mass.).

The precursor molecule may also include any precursor for post-translational or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non essential amino acid is known —e.g., 4 in alanine, 2 in glycine, etc.

The precursor molecule for proteins may be water. The hydrogen atoms on C—H bonds are the hydrogen atoms on amino acids that are useful for measuring protein synthesis from ²H₂O since the O—H and N—H bonds of peptides and proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of H-label in C—H bonds of protein-bound amino acids after ²H₂O administration therefore means that the protein was assembled from amino acids that were in the free form during the period of 2H₂O exposure—i.e., that the protein is newly synthesized. Analytically, the amino acid derivative used must contain all the C—H bonds but must remove all potentially contaminating N—H and O—H bonds.

Hydrogen atoms from body water may be incorporated into free amino acids. ²H or ³H from labeled water can enter into free amino acids in the cell through the reactions of intermediary metabolism, but ²H or ³H cannot enter into amino acids that are present in peptide bonds or that are bound to transfer RNA. Free essential amino acids may incorporate a single hydrogen atom from body water into the a-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutrate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histine, the hydroxyl group in hydroxyproline or hydroxylysine, and others. Other amino acids synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme catalyzed reactions. Incorporation of labeled oxygen into amino acids is known to one of skill in the art. Oxygen atoms may also be incorporated into amino acids from ¹⁸O₂ through enzyme catalyzed reactions (including hydroxyproline, hydroxylysine or other post-translationally modified amino acids).

Hydrogen and oxygen labels from labeled water may also be incorporated into amino acids through post-translational modifications. In one embodiment, the post-translational modification may already include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange labeled hydrogens from body water, either before or after post-translational modification step (e.g. methylation, hydroxylation, phosphoryllation, prenylation, sulfation, carboxylation, acetylation or other known post-translational modifications).

(b) Precursors of Oligo/Polynucleotides

The precursor molecule may include components of oligo or polynucleotides (oligonucleotide and polynucleotide used interchangeably in this context). Polynucleotides include purine and pyrimidine bases and a ribose-phosphate backbone. The precursor molecule may be any polynucleotide precursor molecule known in the art.

The precursor molecules of polynucleotides may be CO₂, NH₃, urea, O₂, glucose, lactate, H₂O, acetate, ketone bodies and fatty acids, glycine, succinate or other amino acids, and phosphate.

Precursor molecules of polynucleotides may also include one or more nucleoside residues. The precursor molecules may also be one or more components of nucleoside residues. Glycine, aspartate, glutamine, and tetryhydrofolate, for example, may be used as precursor molecules of purine rings. Carbamyl phosphate and aspartate, for example, may be used as precursor molecules of pyrimidine rings. Adenine, adenosine, guanine, guanosine, cytidine, cytosine, thymine, or thymidine may be given as precursor molecules for deoxyribonucleosides. All isotope labeled precursors may be purchased commercially, for example, from Cambridge Isotope Labs (Andover, Mass.).

The precursor molecule of polynucleotides may be water. The hydrogen atoms on C—H bonds of polynucleotides, polynucleosides, and nucleotide or nucleoside precursors may be used to measure polynucleotide synthesis from ²H₂O. C—H bonds undergo exchange from H₂O into polynucleotide precursors. The presence of ²H-label in C—H bonds of polynucleotides, nucleosides, and nucleotide or nucleoside precursors, after ²H₂O administration therefore means that the polynucleotide was synthesized during this period. The degree of labeling present may be determined experimentally, or assumed based on the number of labeling sites in a polynucleotide or nucleoside.

Hydrogen atoms from body water may be incorporated into free nucleosides or polynucleotides. ²H or ³H from labeled water can enter these molecules through the reactions of intermediary metabolism.

One of skill in the art will recognize that labeled hydrogen atoms from body water may be incorporated into other polynucleotides, nucleotides, or nucleosides via various biochemical pathways. For example, glycine, aspartate, glutamine, and tetryhydrofolate, which are known precursor molecules of purine rings. Carbamyl phosphate and aspartate, for example, are known precursor molecules of pyrimidine rings. Ribose and ribose phosphate, and their synthesis pathways, are known precursors of polynucleotide synthesis.

Oxygen atoms (H₂¹⁸O) may also be incorporated into polynucleotides, nucleotides, or nucleosides through enzyme-catalyzed biochemical reactions, including those listed above. Oxygen atoms from ¹⁸O₂ may also be incorporated into nucleotides by oxidative reactions, including non-enzymatic oxidation reactions (including oxidative damage, such as formation of 8-oxo-guanine and other oxidized bases or nucleotides).

Isotope-labeled precursors may also be incorporated into polynucleotides, nucleotides, or nucleosides in post-replication modifications. Post-replication modifications include modifications that occur after synthesis of DNA molecules. The metabolic derivatives may be methylated bases, including, but not limited to, methylated cytosine. The metabolic derivatives may also be oxidatively modified bases, including, but not limited to, 8-oxo-guanosine. Those of skill in the art will readily appreciate that the label may be incorporated during synthesis of the modification.

(c) Precursors of Lipids

Labeled precursors of lipids may include any precursor in lipid biosynthesis. The precursor molecules of lipids may be CO₂, NH₃, glucose, lactate, H₂O, acetate, and fatty acids. The precursor may also include labeled water, preferably ²H₂O (deuterated water), which is a precursor for fatty acids, glycerol moiety of acyl-glycerols, cholesterol and its derivatives; ¹³C or ²H-labeled fatty acids, which are precursors for triglycerides, phospholipids, cholesterol ester, coamides and other lipids; ¹³C- or ²H-acetate, which is a precursor for fatty acids and cholesterol; ¹⁸O₂, which is a precursor for fatty acids, cholesterol, acyl-glycerides, and certain oxidatively modified fatty acids (such as peroxides) by either enzymatically catalyzed reactions or by non-enzymatic oxidative damage (e.g. to fatty acids); ¹³C- or ²H-glycerol, which is a precursor for acyl-glycerides; ¹³C- or ²H-labeled acetate, ethanol, ketone bodies or fatty acids, which are precursors for endogenously synthesized fatty acids, cholesterol and acylglycerides; and ²H or ¹³C-labeled cholesterol or its derivatives (including bile acids and steroid hormones). All isotope labeled precursors may be purchased commercially, for example, from Cambridge Isotope Labs (Andover, Mass.).

Complex lipids, such as glycolipids and cerebrosides, can also be labeled from precursors, including ²H₂O, which is a precursor for the sugar-moiety of cerebrosides (including, but not limited to, N-acetylgalactosamine, N-acetylglucosamine-sulfate, glucuronic acid, and glucuronic acid-sulfate), the fatty acyl-moiety of cerebrosides and the sphingosine moiety of cerebrosides; ²H- or ¹³C-labeled fatty acids, which are precursors for the fatty acyl moiety of cerebrosides, glycolipids and other derivatives.

The precursor molecule may be or include components of lipids.

(d) Precursors of Glycosaminoglycans and Proteoglycans

Glycosaminoglycans and proteoglycans are a complex class of biomolecules that play important roles in the extracellular space (e.g. cartilage, ground substance, and synovial joint fluid). Molecules in these classes include, for example, the large polymers built from glycosaminoglycans disaccharides, such as hyaluronan, which is a polymer composed of up to 50,000 repeating units of hyaluronic acid (HA) disaccharide, a dimer that contains N-acetyl-glucosamine linked to glucuronic acid; chondroitin-sulfate (CS) polymers, which are built from repeating units of CS disaccharide, a dimer that contains N-acetyl-galactosamine-sulfate linked to glucuronic acid, heparan-sulfate polymers, which are built from repeating units of heparan-sulfate, a dimer of N-acetyl (or N-sulfo)-glucosamine-sulfate linked to glucuronic acid; and keratan-sulfate polymers, which are built from repeating units of keratan-sulfate disaccharide, a dimer that contains N-acetylglucosamine-sulfate liked to galactose. Proteoglycans contain additional proteins that are bound to a central hyaluronan in polymer and other glycosaminoglycans, such as CS, that branch off of the central hyaluronan chain.

Labeled precursors of glycosaminoglycans and proteoglycans include, but are not limited to, ²H₂O (incorporated into the sugar moieties, including N-acetylglucosamine, N-acetylgalactosamine, glucuronic acid, the various sulfates of N-acetylglucosamine and N-acetylgalactosamine, galactose, iduronic acid, and others), ¹³C- or ²H-glucose (incorporated into sugar moieties), ²H- or ³C-fructose (incorporated into the sugar moieties), ²H- or C-galactose (incorporated into said sugar moieties), ¹⁵N-glycine, other ¹⁵N-labeled amino acids, or ¹⁵N-urea (incorporated into the nitrogen-moiety of the amino sugars, such as N-acetylglycosamine, N-acetyl-galactosamine, etc.); ¹³C- or ²H-fatty acids, ¹³C- or ²H-ketone bodies, ¹³C-glucose, ¹³C-fructose, ¹⁸O₂, ¹³C- or ²H-acetate (incorporated into the acetyl moiety of N-acetyl-sugars, such as N-acetyl-glucosamine or N-acetyl-galactosamine), and ¹⁸O or ³⁵S-labeled sulfate (incorporated into the sulfate moiety of chondroitin-sulfate, heparan-sulfate, keratan-sulfate, and other sulfate moieties). All isotope labeled precursors may be purchased commercially, for example, from Cambridge Isotope Labs (Andover, Mass.).

(e) Precursors of Carbohydrates

Labeled precursors of carbohydrates may include any precursor of carbohydrate biosynthesis known in the art. These precursor molecules include but are not limited to H₂O, monosaccharides (including glucose, galactose, mannose, fucose, glucuronic acid, glucosamine and its derivatives, galactosamine and its derivatives, iduronic acid, fructose, ribose, deoxyribose, sialic acid, erythrose, sorbitol, adols, and polyols), fatty acids, acetate, ketone bodies, ethanol, lactate, alanine, serine, glutamine and other glucogenic amino acids, glycerol, O₂, CO₂, urea, starches, disaccharides (including sucrose, lactose, and others), glucose polymers and other polymers of the monosaccharides (including complex polysaccharides).

The precursor molecule may include labeled water, preferably ²H₂O, which is a precursor to the monosaccharides, ¹³C-labeled glucogenic precursors (including glycerol, CO₂, glucogenic amino acids, lactate, ethanol, acetate, ketone bodies and fatty acids), ¹³C- or ²H-labeled the monosaccharides, ¹³C- or ²H-labeled starches or disaccharides; other components of carbohydrates labeled with ²H or ¹³C; and ¹⁸O₂, which is a precursor to monosaccharides and complex polysaccharides.

2. Methods of Administering labeled precursor molecules

Labeled precursors can be administered to a living system by various in vivo methods including, but not limited to, orally, parenterally, subcutaneously, intravenously, and intraperitoneally.

The living system may be an animal. The living system also may be human.

By way of example, in one embodiment, the labeled precursor is ²H₂O that can be ingested (e.g., by drinking or intravenous infusion) by a living system. In another embodiment, the labeled precursor is ¹³C₁-lysine that can be ingested (e.g., by drinking or intravenous infusion) by a living system. In another embodiment, the labeled precursor is ¹³C₁-glycine that can be ingested (e.g., by drinking or intravenous infusion) by a living system. In another embodiment, the labeled precursor is ²H₃-leucine that can be ingested (e.g., by drinking or intravenous infusion) by a living system. In another embodiment, the labeled precursor is ²H₂-glucose that can be ingested (e.g., by drinking or intravenous infusion) by a living system.

The length of time for which the labeled precursor is administered may be sufficient to allow the precursor molecule to become incorporated into a biosynthetic pathway. The isotope-labeled precursor molecule also may be introduced to a living system for a period of time sufficient for the label of the isotope-labeled precursor molecule to become incorporated into one or more mitochondrial molecules and then released in the form of one or more labeled and unlabeled metabolic derivatives of the one or more mitochondrial molecules. The period of time may be a pre-determined length of time. This required duration of time may range from minutes or hours (e.g., for fast turnover mitochondrial molecules), to weeks or even months (e.g., for slow-turnover mitochondrial molecules).

The precursor molecule may be continuously or repeatedly administered. Administration of the precursor can be achieved in various ways. The precursor molecule may be administered continuously or repeatedly, so that a sufficient amount of precursor is administered such that an isotopic plateau value of maximal or isotopic enrichment is approached (i.e., wherein the concentration of labeled precursor is relatively constant over time). If the continuous labeling period can be maintained for as long as 4-5 half-lives of a mitochondrial molecule, the asymptote reached and the shape of the isotope enrichment or content curve approaching this asymptote will reveal the “true precursor” isotopic enrichment or content as well as the fractional replacement rate of the mitochondrial molecule product. By labeling to plateau while maintaining a stable precursor pool enrichment, it is thereby possible to overcome the biological complexities of cellular metabolite pools.

The precursor molecule may be administered discontinuously. For the discontinuous labeling method, an amount of labeled precursor molecule is measured and then administered, one or more times, and then the exposure to labeled precursor molecule is discontinued and wash-out of labeled precursor molecule from body precursor pool is allowed to occur. The time course of mitochondrial molecule breakdown may then be monitored by measurement of the loss of label or decay of label incorporation (dilution or die-away) in the metabolic derivative of the biological sample.

After administration of isotopically labeled water or other isotopically labeled precursor subunit molecules to a subject, the isotope is generally incorporated into a mitochondrial molecule. Examples of mitochondrial molecules include, but are not limited to, DNA, RNA, proteins, and lipids (e.g., phospholipids).

The methods of this invention are typically carried out in mammalian subjects, preferably humans. Mammals include, but are not limited to, primates, farm animals, sport animals, mice, and rats. If desired, however, the isotopically labeled precursor subunit molecule (including labeled water) may be used in an in vitro system, e.g., to contact a culture of cells or tissue. In this variation, the method for assessing metabolic fitness of the cultured cells or tissue includes: 1) contacting the cell or tissue with labeled water or other isotopically labeled precursor subunit; 2) allowing sufficient time for the label to be incorporated into a newly synthesized mitochondrial molecule; 3) isolating the mitochondria and/or a mitochondrial molecule from the cultured cell or tissue; 4) measuring isotopic content and/or pattern or rate of change of isotopic content and/or pattern of the mitochondrial molecule; and 5) calculating the rate of synthesis or rate of degradation of the mitochondrial molecule.

The labeled water or other isotopically labeled precursor subunits are generally administered at a predetermined volume and isotope concentration. Isotope concentration typically varies depending on the purpose, e.g., initiating the administration protocol of the isotopically labeled precursor subunit (i.e., “priming” the subject) or maintenance of the administration protocol of the isotopically labeled precursor subunit (i.e., “constant administration” to the subject). When given as a primer, deuterated water, for example, may be administered to achieve a sufficient concentration range in body water. Additionally, for maintenance purposes, water, including deuterated water, may be administered as a daily dose (e.g., 70 mL per day) or as a proportion of drinking water (e.g., 4% ²H₂O in drinking water). The labeled water or other isotopically labeled precursor subunit is optionally administered for a duration of time sufficient to achieve relatively stable or constant levels over the time period of incorporation (i.e., steady-state levels) in the cells, tissue, or organism of interest.

The administration of labeled water or other isotopically labeled precursor subunit to subjects may be orally or by parenteral routes, e.g., intravascular infusion or subcutaneous, intramuscular, or intraperitoneal injection.

### B. Obtaining One or More Mitochondrial Molecules

After labeled water or an isotopically labeled precursor subunit has been administered, mitochondria are isolated from one or more cell types or one or more tissue samples of interest, by techniques well known in the art (see Collins M L, Eng S, Hoh R, Hellerstein M K. J Appl Physiol. 2003 June; 94(6):2203-1 1, herein incorporated by reference). Preferably, mitochondria are isolated from blood cells, e.g., platelets or white blood cells such as granulocytes and lymphocytes, or tissue such as skeletal or cardiac muscle. When isolated from blood cells, the cells may be obtained by methods such as venipuncture or needle aspiration, but is not so limited. In addition, tissue samples may be obtained by techniques including, but not limited to, needle aspiration, needle biopsy, endoscopic biopsy, open biopsy, and other surgical biopsy procedures known in art.

If necessary, the mitochondrial molecule (e.g., DNA) is converted to a form in which isotopic content and/or pattern can be measured. The isotopic content and/or pattern of the mitochondrial molecule is then determined by methods including, but not limited to, mass spectrometry, nuclear magnetic resonance spectroscopy, near infra-red laser spectroscopy, liquid scintillation counting or other methods known in the field. Optimally, the isotopic content and/or pattern in the mitochondrial molecule is compared to a reference value representing the isotopic content and/or pattern in the biosynthetic precursor pool, from which the mitochondrial molecule was synthesized in the cell, tissue, or organism. The rate of synthesis of the mitochondrial molecule may then be calculated, as described by Hellerstein et al. (1999), supra, which is herein incorporated by reference in its entirety, based on isotopic content and/or pattern and duration of exposure to the isotopically labeled precursor subunit, after correction for the isotopic content and/or pattern in the biosynthetic precursor pool, according to the precursor-product relationship; or, the rate of degradation of the mitochondrial component may be calculated, based on the time course of die-away of the isotopic content and/or pattern in the mitochondrial molecule after removal or wash-out (i.e., “chase”) of the labeled precursor subunit. The calculated rate(s) of synthesis and/or degradation of mitochondrial molecules may then be used to represent the metabolic fitness of the cell(s) or tissue(s) analyzed.

In practicing the methods of the invention, in one aspect, targeted molecules of interest are obtained from a cell, tissue, or organism according to methods known in the art. The methods may be specific to the particular mitochondrial molecule. Molecules of interest may be isolated from a biological sample.

A plurality of molecules of interest may be acquired from the cell, tissue, or organism. The one or more biological samples may be obtained, for example, by blood draw, urine collection, biopsy, or other methods known in the art. The one or more biological sample may be one or more biological fluids. The mitochondrial molecule may also be obtained from specific organs or tissues, such as muscle, liver, adrenal tissue, prostate tissue, endometrial tissue, blood, skin, and breast tissue. Molecules of interest may be obtained from a specific group of cells, such as tumor cells or fibroblast cells. Molecules of interest also may be obtained, and optionally partially purified or isolated, from the biological sample using standard biochemical methods known in the art.

The frequency of biological sampling can vary depending on different factors. Such factors include, but are not limited to, the nature of the molecules of interest, ease and safety of sampling, synthesis and breakdown/removal rates of the mitochondrial molecule, and the half-life of a chemical entity or drug agent.

The molecules of interest may also be purified partially, or optionally, isolated, by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the molecules of interest. The molecules of interest also may be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

### C. Biochemical Analysis

Presently available technologies (static methods) used to identify biological actions of agents measure only composition, structure, or concentrations of molecules in a cell or subcellular organelle (e.g., a mitochondrion) and do so at one point in time. The methods of the present invention, however, allow determination of the molecular flux rates of mitochondrial molecules (e.g., DNA, RNA, proteins, lipids) and their changes over time in a variety of disease states and in response to formal or informal exercise, specific training regimens, inactivity, bed-rest, life-style changes, or other behavioral factors or to exposure to an agent or combination of agents. This allows for a more accurate assessment of a living system's fitness state (i.e., metabolic fitness) and/or aerobic capacity under a broad spectrum of physiological and pharmacological conditions as the synthesis or degradation of a mitochondrial molecule can be accomplished and a direct assessment of mitochondrial biogenesis can therefore be made. In contrast, a pure static measurement of mitochondrial molecules provides little useful information in assessing mitochondrial biogenesis and consequently is of little practical value in assessing a living system's fitness state (i.e., metabolic fitness) and/or aerobic capacity.

1. Mass Spectrometry

Isotopic enrichment in mitochondrial molecules can be determined by various methods such as mass spectrometry, including but not limited to gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix assisted laser desorption-time of flight-MS, Fourier-transform-ion-cyclotron-resonance-MS, and cycloidal-MS.

Mass spectrometers convert molecules such as proteins, lipids, carbohydrates, nucleic acids, and organic metabolites into rapidly moving gaseous ions and separate them on the basis of their mass-to-charge ratios. The distributions of isotopes or isotopologues of ions, or ion fragments, may thus be used to measure the isotopic enrichment in a plurality of mitochondrial molecules.

Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, two or more mass analyzers may be coupled (MS/MS) first to separate precursor ions, then to separate and measure gas phase fragment ions. These instruments generate an initial series of ionic fragments of a protein, and-then generate secondary fragments of the initial ions. The resulting overlapping sequences allows complete sequencing of the protein, by piecing together overlaying “pieces of the puzzle”, based on a single mass spectrometric analysis within a few minutes (plus computer analysis time).

The MS/MS peptide fragmentation patterns and peptide exact molecular mass determinations generated by protein mass spectrometry provide unique information regarding the amino acid sequence of proteins and find use in the present invention. An unknown protein can be sequenced and identified in minutes, by a single mass spectrometric analytic run. The library of peptide sequences and protein fragmentation patterns that is now available provides the opportunity to identify components of complex mixtures with near certainty.

Different ionization methods are also known in the art. One key advance has been the development of techniques for ionization of large, non-volatile macromolecules including proteins and polynucleotides. Techniques of this type have included electrospray ionization (ESI) and matrix assisted laser desorption (MALDI). These have allowed MS to be applied in combination with powerful sample separation introduction techniques, such as liquid chromatography and capillary zone electrophoresis.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS (or other mass spectrometric modalities that analyze ions of proteins, nucleic acids, lipids, and organic metabolites, rather than small inorganic gases) is used to measure mass isotopomer abundances of organic molecules, hydrogen-labeled isotope incorporation from isotope-labeled water is amplified 3 to 7-fold, depending on the number of hydrogen atoms incorporated into the organic molecule from isotope-labeled water in vivo.

In general, in order to determine a baseline mass isotopomer frequency distribution for the mitochondrial molecule, such a sample is taken before infusion of an isotopically labeled precursor. Such a measurement is one means of establishing in the cell, tissue or organism, the naturally occurring frequency of mass isotopomers of the mitochondrial molecule. When a cell, tissue or organism is part of a population of subjects having similar environmental histories, a population isotopomer frequency distribution may be used for such a background measurement. Additionally, such a baseline isotopomer frequency distribution may be estimated, using known average natural abundances of isotopes. For example, in nature, the natural abundance of ¹³C present in organic carbon is 1.11%. Methods of determining such isotopomer frequency distributions are discussed below. Typically, samples of the mitochondrial molecule are taken prior to and following administration of an isotopically labeled precursor to the subject and analyzed for isotopomer frequency as described below.

1. Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

2. Calculating Labeled: Unlabeled Proportion of Molecules of Interest

The proportion of labeled and unlabeled molecules of interest is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value Aₓ*, which describes the enrichment of newly synthesized molecules for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer Mₓ,

$$Fractional abundance of {M}_{x} = {A}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

$$\begin{matrix} \begin{matrix}Δ Fractional abundance \\ ( enrichment or depletion )\end{matrix} = {( {A}_{x} )}_{e} - {( {A}_{x} )}_{b} \\ = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,\end{matrix}$$

where subscript e refers to enriched and b refers to baseline or natural abundance.

In order to determine the fraction of the molecules that were actually newly synthesized during a period of precursor administration, the measured excess molar ratio (EMₓ) is compared to the calculated enrichment value, Aₓ*, which describes the enrichment of newly synthesized biopolymers for each mass isotopomer, to reveal the isotopomer excess ratio which would be expected to be present, if all isotopomers were newly synthesized.

3. Calculating Molecular Flux Rates

The method of determining rate of synthesis includes calculating the proportion of mass isotopically labeled subunit present in the molecular precursor pool, and using this proportion to calculate an expected frequency of a molecule of interest containing at least one mass isotopically labeled subunit. This expected frequency is then compared to the actual, experimentally determined isotopomer frequency of the molecule of interest. From these values, the proportion of the molecule of interest which is synthesized from added isotopically labeled precursors during a selected incorporation period can be determined. Thus, the rate of synthesis during such a time period is also determined.

A precursor-product relationship may then be applied. For the continuous labeling method, the isotopic enrichment is compared to asymptotic (i.e., maximal possible) enrichment and kinetic parameters (e.g., synthesis rates) are calculated from precursor-product equations. The fractional synthesis rate (kₛ) may be determined by applying the continuous labeling, precursor-product formula:

kₛ=[−1n(1−f)]/t,

where f=fractional synthesis=product enrichment/asymptotic precursor/enrichment and t=time of label administration of contacting in the system studied.

For the discontinuous labeling method, the rate of decline in isotope enrichment is calculated and the kinetic parameters of the molecules of interest are calculated from exponential decay equations. In practicing the method, biopolymers are enriched in mass isotopomers, preferably containing multiple mass isotopically labeled precursors. These higher mass isotopomers of the molecules of interest, e.g., molecules containing 3 or 4 mass isotopically labeled precursors, are formed in negligible amounts in the absence of exogenous precursor, due to the relatively low abundance of natural mass isotopically labeled precursor, but are formed in significant amounts during the period of molecular precursor incorporation. The molecules of interest taken from the cell, tissue, or organism at the sequential time points are analyzed by mass spectrometry, to determine the relative frequencies of a high mass isotopomer. Since the high mass isotopomer is synthesized almost exclusively before the first time point, its decay between the two time points provides a direct measure of the rate of decay of the molecule of interest.

Preferably, the first time point is long enough after administration of precursor has ceased, depending on mode of administration, to ensure that the proportion of mass isotopically labeled subunit has decayed substantially from its highest level following precursor administration. In one embodiment, the following time points are typically 1-4 hours after the first time point, but this timing will depend upon the replacement rate of the biopolymer pool.

The rate of decay of the molecule of interest is determined from the decay curve for the three-isotope molecule of interest. In the present case, where the decay curve is defined by several time points, the decay kinetic can be determined by fitting the curve to an exponential decay curve, and from this, determining a decay constant.

Breakdown rate constants (k_{d}) may be calculated based on an exponential or other kinetic decay curve:

k_{d}=[−1n f]/t.

As described, the method can be used to determine subunit pool composition and rates of synthesis and decay for substantially any biopolymer which is formed from two or more identical subunits which can be mass isotopically labeled. Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest.

### Applications of the Inventive Methods

The methods of the present invention may be used for a variety of purposes. Primarily, the methods are used to assess the metabolic fitness of a subject. In turn, the metabolic fitness of the subject may be used to determine the risk of that subject for medical conditions such as cardiovascular disease and diabetes mellitus, or for mortality in general. Once a particular risk has been assessed, appropriate treatment can be recommended.

In another variation, the methods may be employed in a subject, cell culture, or tissue culture to screen drug agents, such as candidate pharmaceutical agents, in a high-throughput manner, for their effect on metabolic fitness (i.e., ability to alter metabolic fitness by increasing or decreasing metabolic fitness, or ability to prevent changes in metabolic fitness). If a cell culture system is used, the methods of the invention can be employed to screen pharmaceutical agents or candidate pharmaceutical agents in a high throughput system. Whether used in vivo or in vitro, the effect on metabolic fitness is determined by measuring and then comparing metabolic fitness before and after administration of the drug/pharmaceutical agent or candidate drug/pharmaceutical agent. The resulting difference in metabolic fitness is the effect which the candidate drug agent has on the subject, cell, or tissue of interest. For example, exercise training generally improves the metabolic fitness of a subject. Subsequent inactivity (detraining or deconditioning) for at least approximately 2 weeks typically results in a decrease in metabolic fitness. However, use of this inventive method would help to identify a drug or candidate drug agent that prevents detraining and thereby has therapeutic utility in people forced to undergo bed-rest due to injury, illness, immobilization, or other change in metabolic fitness or aerobic demand.

The effect of a drug agent may be tested using the methods described herein. A change in the metabolic fitness or aerobic demand of a living system to which a drug agent has been administered and a living system to which a drug has not been administered identifies the drug agent as capable of altering metabolic fitness or aerobic demand of a living system. The drug agent may be administered to the same living system, or different living systems. Drug agents may be any chemical compound or composition known in the art. Drug agents include, but are not limited to, any chemical compound or composition disclosed in, for example, the 13th Edition of The Merck Index (a U.S. publication, Whitehouse Station, N.J., U.S.A.), incorporated herein by reference in its entirety.

In a further variation, the invention provides kits for performing the methods of the invention. The kits may be formed to include such components as labeled water, one or more other isotopically labeled precursor subunits, or mixtures thereof. The labeled water or other isotopically labeled precursor subunit(s) may be supplied in varying isotope concentrations and as premeasured volumes. Furthermore, the kits preferably will be packaged with instructions for use of the kit components and with instructions on how to calculate metabolic fitness.

Other kit components, such as tools for administration of labeled water or an isotopically labeled precursor subunit (e.g., measuring cup, needles, syringes, pipettes, IV tubing), may optionally be provided in the kits. Similarly, instruments for obtaining samples from the subject, cell, or tissue culture (e.g., scalpel, forceps, needles, syringes, and vacutainers) may also be optionally provided.

The following examples are provided to show that the methods of the invention may be used to assess metabolic fitness of cells, tissues, or organisms, including humans. Those skilled in the art will recognize that while specific embodiments have been illustrated and described, they are not intended to limit the invention.

### EXAMPLES

### Example 1

### Fractional Synthesis of Mitochondrial DNA in Rats After Isotopically Labeled Water Administration

The protocol for incorporation of ²H into rat mitochondrial DNA is illustrated in the experimental design of FIG. 1A. Male Sprague Dawley rats from Simonsen, Inc. Gilroy, Calif. were primed with 100% ²H₂O via intraperitoneal injection (a) on day zero to achieve 2% ²H₂O in body water of the rats. Deuterated water (4% ²H₂O) was then administered as drinking water to the rodents for about 10 weeks (b). There were two groups of rats:trained and untrained. The animals were then sacrificed at various timepoints (c), and tissue samples obtained from cardiac and hindlimb muscle. Thereafter, mitochondria were collected by centrifugation and mitochondrial DNA was isolated using ultracentrifugation and biochemical isolation techniques well known in the art (see Collins M L, Eng S, Hoh R, Hellerstein M K. J Appl Physiol. 2003 June; 94(6):2203-11). The DNA was hydrolyzed to free deoxyribonucleosides and derivatized using techniques known in the art (Collins et al., supra).

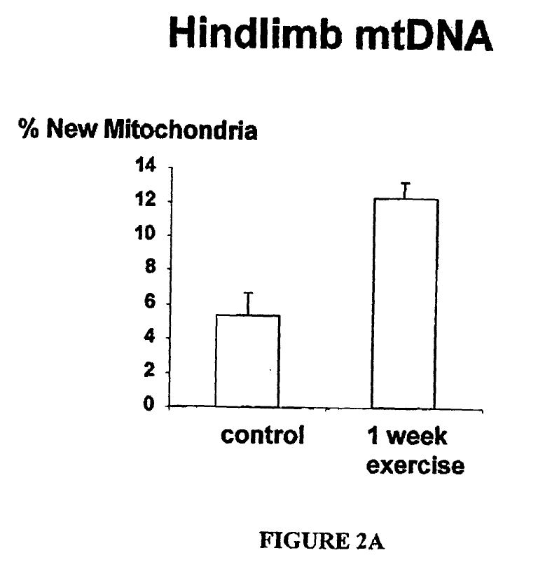

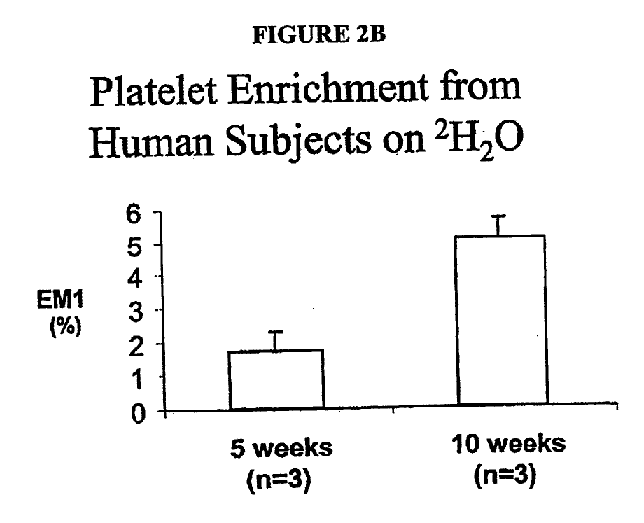

As shown in FIG. 2A, the incorporation of ²H into mitochondrial DNA was measured by gas chromatography/mass spectrometry. Animals placed on an exercise training (treadmill running) program for 1 week of exercise exhibited markedly increased incorporation of ²H into mitochondrial DNA. Conversely, sedentary, obese mice exhibited reduced mitochondrial DNA synthesis.

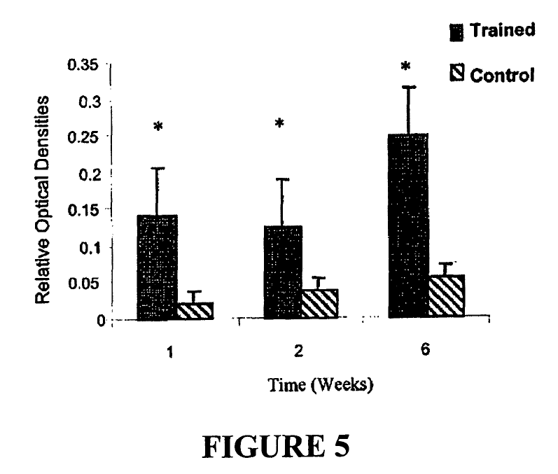

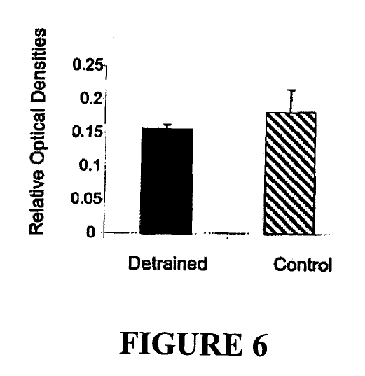

Cytochrome c oxidase subunit IV content increased with training (FIG. 5) and returned to sedentary control levels with detraining (FIG. 6). After 4 weeks of detraining, cytochrome C oxidase content in the previously trained group was not significantly different from sedentary control values, 0.15±0.01 and 0.17±0.04 mean relative optical intensities, respectively (FIG. 6). Because subunit IV is not coded by mtDNA, synthesis of new mtDNA can not directly lead to increased subunit IV content and subunit IV content does not directly represent mtDNA replication or transcription. The coordinate increases in mtDNA synthesis and subunit IV content that we observed here in both training and detraining are consistent with shared regulation by nuclear and mitochondrial elements (Williams et al., 1986). Also, if the ratio between oxidative enzymes and mtDNA content remains relatively constant in the mitochondria of a cell (Williams et al., 1986), the subinit IV content can be used as a marker of tissue mitochondrial mass, to allow fractional mtDNA synthesis to be converted to absolute biogenesis rates. Application of this technique has potential advantages over measurement of cytochrome c oxidase levels alone, since kinetic changes typically precede and are more sensitive than changes in static measures.

### Example 2

### Fractional Synthesis ofMitochondrial DNA Isolated From Human Blood Platelets After Isotopically Labeled Water Administration

The protocol for incorporation of ²H into human mitochondrial DNA from blood platelets is illustrated in the experimental design of FIG. 1B. Human subjects from the General Clinical Research Center of San Francisco General Hospital were primed with 560 ml of 70% ²H₂O by drinking 70 mls every three hours over 24 hours (a) at day zero and given 150 ml of 70% ²H₂O by drinking 50 mls 3 times a day for about 11 days. A volume of 70 ml/day of 70% ²H₂O was then administered by drinking 35 mls 2 times a day for about the next 10 weeks. Blood was drawn at various timepoints (c) and platelets isolated from the samples.

FIG. 2B shows that enrichment of platelet mitochondrial DNA from deuterated water administration increases with the increasing duration of administration of ²H₂O (Collins et al., supra).

### Example 3

### Fractional Synthesis ofMitochondrial DNA and Phospholipids Isolated From Human Muscle Biopsies After Isotopically Labeled Water Administration

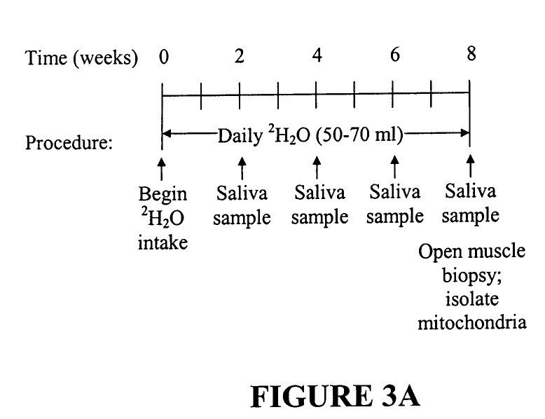

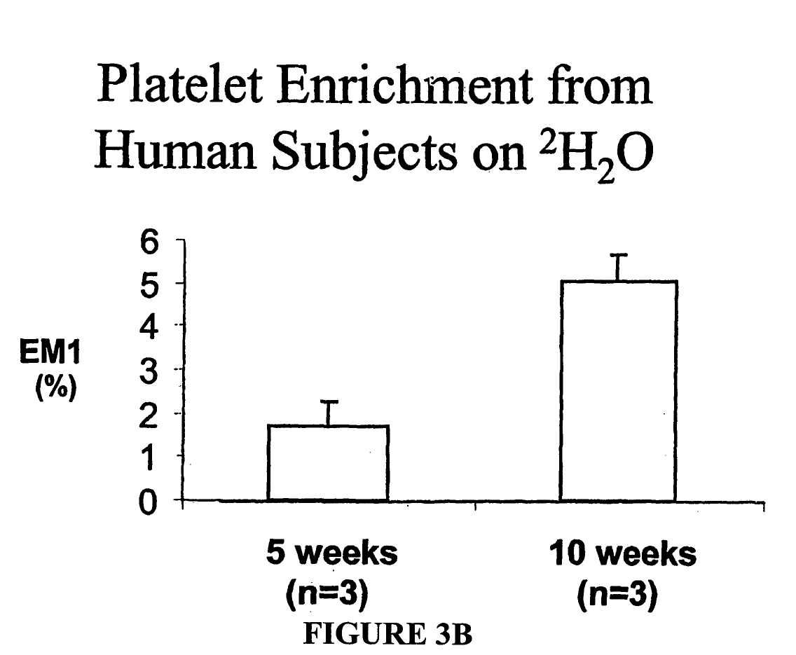

The protocol for incorporation of ²H into human mitochondrial DNA from muscle biopsy samples is illustrated in the experimental design of FIG. 3d. Five human subjects enrolled as out-patients ingested 70 ml of 70% ²H₂O three times a day for 5 days then twice a day for 5 days; then ingested 50 ml twice a day thereafter for the remainder of the eight-week study period. Every two weeks, subjects gave a saliva sample (for measurement of body 2H₂O enrichment). At week 8, an open muscle biopsy was performed under surgical conditions. Mitochondria were isolated from excised muscle tissue (1 g) by ultracentrifugation, using methods well known in the art. Isolation of mitochondrial (mt) DNA and phospholipids (PL) were by procedures well known in the art. Measurement of fractional synthesis of mt PL were as described in the general methods, supra and in Collins et al., supra. Measured ²H-incorporation (EM₁=excess abundance of M+1 mass isotopomer of the molecule) and fractional synthesis (f) of mt DNA and mt PL are shown in Table 1.

Mitochondrial Phospholipid

| | Mitochondrial DNA | | Cardiolipin | | Phosphatidylcholine | | |
|---|---|---|---|---|---|---|---|
| Subject# | EM₁ | f(%) | EM₁ | f(%) | EM₁ | f(%) | Body ²H₂O (%) |
| 251498 | 0.29 | 21.1 | 1.68 | 97 | 1.83 | 100 | 0.5 |
| 251515 | 0.13 | 2.8 | 1.08 | 19 | 0.44 | 8 | 1.7 |
| 251598 | 0.10 | 3.3 | 2.12 | 58 | 2.38 | 65 | 1.1 |
| 251748 | 0.17 | 3.1 | 3.18 | 49 | 3.40 | 53 | 2.0 |
| 251771 | 0.03 | 1.1 | 1.14 | 34 | 1.46 | 44 | 1.0 |

Variability in fractional synthesis of mt DNA and mt PL is apparent among healthy subjects and may reflect differences in exercise patterns or muscle aerobic demands. Different values for mt DNA and mt PL may reflect differential turnover of different components of human mitochondria. Ratios between mt DNA and mt PL synthesis may also provide information about exercise patterns or tissue aerobic demands.

### Example 4

### Fractional Synthesis of Mitochondrial Phospholipids in Rats After Isotopically Labeled Water Administration

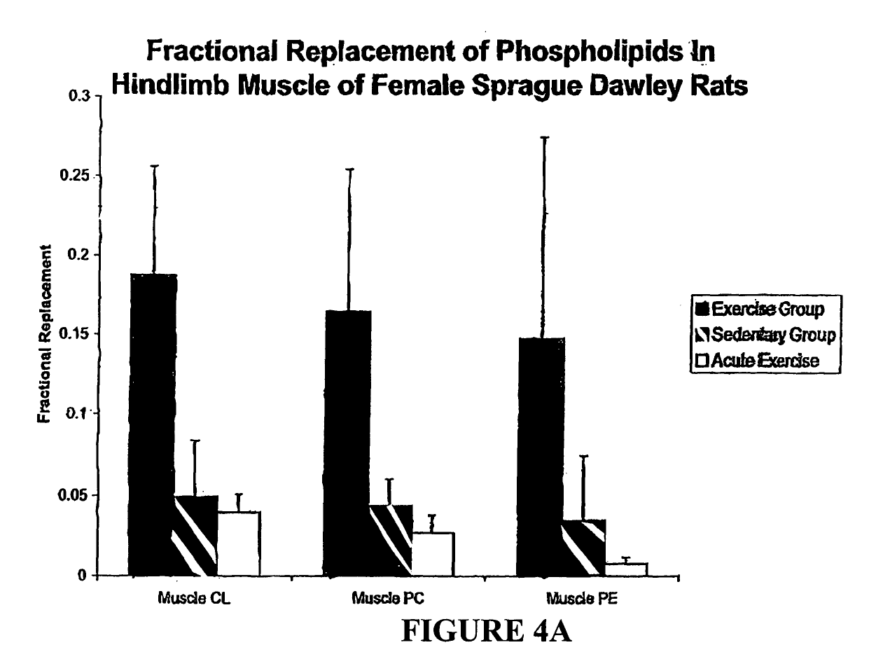

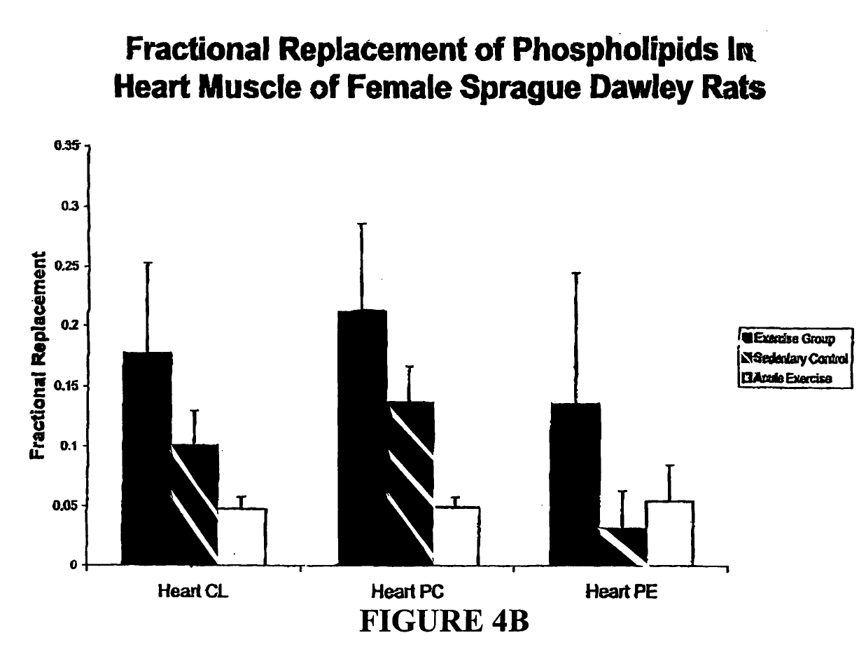

The protocol for incorporation of ²H into rat mitochondrial phospholipids is illustrated in the experimental design of FIG. 4. Female Sprague Dawley Rats from Simonsen, Inc. Gilroy, Calif. were placed into three groups, a trained group, a sedentary control, and an acute exercise group. The terms “run” and “exercise” are used synonymously in FIG. 4. The rats were primed and maintained on 4% ²H₂O as described in Example 1. After 57 days, the animals were sacrificed and tissue samples obtained from either the hindlimb muscle or cardiac muscle. Mitochondria were isolated as previously described, and assays for fractional synthesis of cardiolipin (CL), phoshphatidylcholine (PC), and phosphatidylethanolamine (PE) performed as described in Example 3, supra.

As shown in FIGS. 4A and 4B, ²H incorporation into CL, PC, and PE was the greatest in the exercise group of animals.

The results of these studies demonstrate that a laboratory test involving the drinking of deuterated water and the measurement of deuterium incorporation into molecules isolated from mitochondria, can replace physiologic/whole-body exercise tests as indices of metabolic fitness and tissue oxidative needs.

All publications, patents, and patent applications cited herein are hereby incorporated by reference in their entirety for all purposes to the same extent as if each individual publication, patent, or patent application were specifically and individually indicated to be so incorporated by reference. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of the appended claims.

Applicants have not abandoned or dedicated to the public any unclaimed subject matter.

I claim:

1. A method for assessing metabolic fitness or aerobic demand of a living system, comprising:
a) administering an isotopically labeled precursor molecule to the living system for a period of time sufficient for the label of said isotopically labeled precursor molecule to be incorporated into a mitochondrial molecule in said living system;
b) measuring the isotopic content, isotopic pattern, rate of change of isotopic content, or rate of change of isotopic pattern of said mitochondrial molecule; and
c) calculating the rate of synthesis or degradation of said mitochondrial molecule to assess metabolic fitness or aerobic demand of said living system.

2. The method of claim 1, wherein the isotopically labeled precursor molecule is labeled with a stable isotope.

3. The method of claim 1, wherein the isotopically labeled precursor is selected from the group consisting of ²H-labeled glucose, ¹³C-labeled glucose, a ²H-labeled amino acid, a ¹⁵N-labeled amino acid, a ¹³C-labeled amino acid, ²H-labeled acetate, ¹³C-labeled acetate, a ²H-labeled ribonucleoside, a ¹³C-labeled ribonucleoside, a ¹⁵N-labeled ribonucleoside, a ²H-labeled deoxyribonucleoside, a ¹³C-labeled deoxyribonucleoside, a ¹⁵N-labeled deoxyribonucleoside, a ²H-labeled fatty acid, and a ¹³C-labeled fatty acid.

4. The method of claim 1, wherein the isotopically labeled precursor molecule is ²H₂O.

5. The method of claim 1 wherein the isotopically labeled precursor molecule is ¹³C-glycine.

6. The method of claim 1, wherein the label of said isotopically labeled precursor is a radioactive isotope.

7. The method of claim 1, wherein the isotopically labeled precursor molecule is selected from the group consisting of ³H-labeled glucose, ¹⁴C-labeled glucose, a ³H-labeled amino acids, a ¹⁴C-labeled amino acid, ³H-labeled acetate, ¹⁴C-labeled acetate, a ³H-labeled ribonucleoside, a ¹⁴C-labeled ribonucleoside, a ³H-labeled deoxyribonucleoside, a ¹⁴C-labeled deoxyribonucleoside, a ³H-labeled fatty acid, and a ¹⁴C-labeled fatty acid.

8. The method of claim 1, wherein the mitochondrial molecule is a deoxyribonucleic acid (DNA).

9. The method of claim 1, wherein the mitochondrial molecule is a ribonucleic acid (RNA).

10. The method of claim 9, wherein the RNA is selected from the group consisting of ribosomal RNA, transfer RNA, and messenger RNA.

11. The method of claim 10, wherein the RNA is messenger RNA.

12. The method of claim 1, wherein the mitochondrial molecule is a protein.

13. The method of claim 12, wherein the protein is selected from the group consisting of a subunit of cytochrome c oxidase, a subunit of F₀ ATPase, a subunit of F₁ ATPase, a subunit of cytochrome c reductase, and a subunit of NADH-CoQ reductase.

14. The method of claim 1, wherein the mitochondrial molecule is a lipid.

15. The method of claim 14, wherein the lipid is a phospholipid.

16. The method of claim 15, wherein the phospholipid is selected from the group consisting of cardiolipin, phosphatidylcholine, phosphatidylethanolamine, and a mixture thereof.

17. The method of claim 1, wherein the living system is a tissue.

18. The method of claim 17, wherein the tissue is muscle.

19. The method of claim 18, wherein the muscle is skeletal muscle or cardiac muscle.

20. The method of claim 17, wherein the tissue is adipose tissue.

21. The method of claim 1, wherein the step of measuring isotopic content, pattern or rate of change of isotopic content, or pattern is performed by mass spectroscopy, NMR spectroscopy, or liquid scintillation counting.

22. The method of claim 1 wherein the isotopically labeled precursor molecule is administered orally.

23. The method of claim 1, wherein the living system is an animal.

24. The method of claim 23, wherein the animal is a mammal.

25. The method of claim 24, wherein the mammal is a rodent.

26. The method of claim 24, wherein the mammal is a human.

27. The method of claim 1, wherein the living system is a cell.

28. The method of claim 27, wherein the cell is a platelet.

29. The method of claim 27, wherein the cell is a cultured cell in a high-throughput screening assay system.

30. A method of identifying a drug agent capable of altering metabolic fitness or aerobic demand of a living system comprising:
a) assessing the metabolic fitness or aerobic demand of the living system according to claim 1;
b) administering the drug agent to said living system; and
c) assessing the metabolic fitness or aerobic demand of the living system according to claim 1, wherein a change in the metabolic fitness or aerobic demand of the living system before and after administration of the drug agent identifies the drug agent as capable of altering the metabolic fitness or aerobic demand of the living system.

31. The method of claim 30, wherein the living system is a mammal.

32. The method of claim 31, wherein the mammal is a human.

33. The method of claim 31, wherein the mammal is a rodent.

34. The method of claim 30, wherein the living system is a cell.

35. The method of claim 34, wherein the cell is a cultured cell in a high-throughput screening assay system.

36. The method of claim 35, wherein the isotopically labeled precursor molecule is contacted with cell culture media.

37. The method of claim 30, wherein the drug agent is tested for the ability to prevent deconditioning of a living system.

38. The method of claim 30, wherein the drug agent is tested for the ability to increase metabolic fitness or aerobic demand in response to an exercise or other training regimen.

39. A method of identifying a drug agent capable of altering metabolic fitness or aerobic demand of a living system comprising:
a) assessing the metabolic fitness or aerobic demand of a first said living system according to claim 1, wherein the drug agent has not been administered to said first living system;
b) assessing the metabolic fitness or aerobic demand of a second said living system according to claim 1, wherein the drug agent has been administered to said second living system;
c) comparing the metabolic fitness or aerobic demand in said first and second living systems, wherein a change in the metabolic fitness or aerobic demand of the first and second living systems identifies the drug agent as capable of altering the metabolic fitness or aerobic demand of the living system.

40. The method of claim 39, wherein the living system is a mammal.

41. The method of claim 40, wherein the mammal is a human.

42. The method of claim 40, wherein the mammal is a rodent.

43. The method of claim 39, wherein the living system is a cell.

44. The method of claim 43, wherein the cell is a cultured cell in a high-throughput screening assay system.

45. The method of claim 44, wherein the isotopically labeled precursor molecule is contacted with cell culture media.

46. The method of claim 39, wherein the drug agent is tested for the ability to prevent deconditioning of a living system.

47. The method of claim 39, wherein the drug agent is tested for the ability to increase metabolic fitness or aerobic demand in response to an exercise or other training regimen.

48. A kit for assessing the metabolic fitness of a living system, comprising:
a) one or more isotopically labeled precursor molecules; and
b) instructions for use of the kit,
wherein the kit is used to measure metabolic fitness.

49. The kit of claim 48, further comprising a tool for administering the isotopically labeled precursor molecule.

50. The kit of claim 48, further comprising an instrument for obtaining a sample from the subject.

51. The kit of claim 48, wherein said isotopically labeled precursor molecule is isotopically labeled water.

52. A drug agent identified by the method of claim 30.

53. A drug agent identified by the method of claim 39.

54. An isolated isotopically perturbed mitochondrial DNA.

55. An isolated isotopically perturbed cardiolipin.

56. One or more isolated isotopically perturbed mitochondrion.

57. An isotope-labeled precursor molecule.

58. An isolated isotope-labeled mitochondrial molecule made by administering an isotope-labeled precursor molecule to said host organism for a period of time sufficient for an isotope label of said isotope-labeled precursor molecule to become incorporated into a mitochondrial molecule.
